(19) United States
(12) Patent Application Publication
(10) Pub. No.: US 2005/0074819 A1
(43) Pub. Date: Apr. 7, 2005

(54) Screening method of drug for treatment of neuropathic pain

(75) Inventors: Kazuhide Inoue, Tokyo-To (JP); Makoto Tsuda, Tokyo-To (JP); Schuichi Koizumi, Tokyo-To (JP); Shinichi Kohsaka, Tokyo-To (JP)

Correspondence Address: LADAS & PARRY, 26 West 61st Street, New York, NY, 10023, US

(73) Assignee: JAPAN HEALTH SCIENCES FOUNDATION

(21) Appl. No.: 10/676,289

(22) Filed: Oct. 1, 2003

(51) Int. Cl. C12Q 1/68; G01N 33/53; G01N 33/567

(52) U.S. Cl. 435/7.2; 435/6; 514/44

(57) ABSTRACT

The present invention relates to a screening method for a compound useful for treatment of neuropathic pain, and therapy for the treatment of neuropathic pain. The screening method comprises determining inhibitory activity on the action of P2X₄ receptor or inhibitory activity on the activation of microglia, of test compounds. The pharmaceutical composition and the therapy employ a P2X₄ receptor inhibitor or a microglial activation-inhibitor as active ingredient.

### FIELD OF THE INVENTION

The present invention relates to a screening method for a compound useful for treatment of neuropathic pain, and a therapy for the treatment of neuropathic pain.

### BACKGROUND ART

P2 receptors, which are activated by ATP (adenosine 5′-triphosphate) and other nucleotides, consist of two families: P2X receptors and P2Y receptors. P2X receptors are ligand-gated cation channels, including seven subtypes: P2X₁, P2X₂, P2X₃, P2X₄, P2X₅, P2X₆, and P2X₇. P2Y receptors are G protein-coupled receptors, including seven subtypes: P2Y₁, P2Y₂, P2Y₄, P2Y₆, P2Y₁₁, P2Y₁₂, and P2Y₁₃.

Some P2X receptors are reported to be involved in pain. P2X₃ homo-receptor is known to be involved in pain induced by ATP-mediated stimulation of peripheral skin, including spontaneous pain, hyperalgesia by heat-stimulation, and nociceptive flexion reflex (Hamilton, S. G. and McMahon, S. B., J. Auton. Nerv. Syst. 81, 187-194, 2000; Tsuda, M. et al., J. Neurosci. 20, RC90, 2000; Ueda, H. et al., Peptides 22, 1215-1221, 2000; Cockayne, D. A. et al., Nature 407, 1011-1015, 2000; Souslova, V. et al., Nature 407, 1015-1017, 2000). In addition, P2X_{2/3} hetero-receptor is known to be involved in allodynia (Tsuda, M. et al., J. Neurosci. 20, RC90, 2000).

P2X₄ receptor (Bo, X. et al., FEBS Lett. 375, 129-133, 1995; Buell, G. et al., EMBO J. 15, 55-62,1996; Seguela, P. et al., J. Neurosci. 16, 448-455, 1996) is known to be widely expressed in brain and peripheral tissues including various endocrine tissues (Wang, C. Z. et al., Biochem. Biophys. Res. Commun. 220, 196-202, 1996). In addition, P2X₄ receptor is known to be ion-channel for Na⁺, K⁺, and Ca²⁺ (Soto, F. et al., Proc. Natl. Acad. Sci. USA 93, 3684-3688,1996). P2X₄ receptor has not been reported to be involved in pain.

Neuropathic pain is an expression of pathological operation of the nervous system, which commonly results from nerve injury (Woolf, C. J. & Mannion, R. J., Lancet 353, 1959-1964, 1999; Woolf, C. J. & Salter, M. W., Science 288, 1765-1769, 2000), and one hallmark of which is tactile allodynia—pain hypersensitivity evoked by innocuous stimuli. In relation to such pain, it has been reported that PPADS (Pyridoxalphosphate-6-azophenyl-2′,4′-disulphonic acid) administration at the incision area suppressed allodynia in a rat model of postoperative pain (Tsuda, M. et al., Neuroreport 12, 1701-1704, 2001). PPADS is an antagonist of P2X_{1,2,3,5,7} receptors but not of P2X₄ receptor. However, PPADS is effective only when administered before surgical operation.

Microglia is one of glia cells, and is known to exhibit ramified form as inactive-form and amoeboid form with ruffling as active-form. Microglia is known to be activated by ATP or ADP through P2Y₁₂ receptor on the surface thereof (Honda S. et al., Extracellular ATP or ADP induce chemotaxis of cultured microglia through Gi/o-coupled P2Y receptors. J. Neurosci. 21, 1975-1982, 2001). Hyperactive microglia are considered crucial for the pathogenesis of various pathological conditions of the CNS, such as neurodegenerative disorders and stroke (Nakajima, K. & Kohsaka, S. Functional roles of microglia in the brain. Neurosci Res 17, 187-203. (1993); Carson, M. J. Microglia as liaisons between the immune and central nervous systems: Functional implications for multiple sclerosis. Glia 40, 218-231. (2002); Eikelenboom, P. et al. Neuroinflammation in Alzheimer's disease and prion disease. Glia 40, 232-239. (2002)). On the other hand, hyperactive microglia are not reported to be involved in neuropathic pain.

Accordingly, effective therapy for neuropathic pain is lacking and the underlying mechanisms are poorly understood.

### SUMMARY OF THE INVENTION

We have found that pharmacological blockade of spinal P2X₄ receptors (also referred to as P2X₄Rs) reversed tactile allodynia caused by peripheral nerve injury, without affecting acute pain behaviours in naive animals. After nerve injury, P2X₄R expression increased strikingly in the ipsilateral spinal cord and P2X₄Rs were induced in hyperactive microglia but not in neurons or astrocytes. Intraspinal administration of P2X₄R antisense reduced induction of P2X₄Rs and suppressed tactile allodynia following nerve injury. Conversely, intraspinal administration of microglia in which P2X₄Rs had been induced and stimulated, produced tactile allodynia in naive rats. Taken together our results demonstrate that activation of P2X₄Rs in hyperactive microglia is necessary for tactile allodynia following nerve injury and is sufficient to produce tactile allodynia in normal animals. Thus, blocking P2X₄Rs in microglia and inhibiting activation of microglia are new therapeutic strategies for nerve injury-induced pain.

In one aspect of the present invention, a method of identifying a compound useful for the treatment or prevention of neuropathic pain is provided, which comprises: (a) contacting a cell expressing P2X₄ receptor on the surface thereof, with a test compound, in the presence of P2X₄ receptor agonist, (b) determining whether or not said test compound inhibits an interaction of said P2X₄ receptor agonist and P2X₄ receptor on the surface of the cell, and (c) identifying the test compound which inhibits said interaction, as useful for the treatment or prevention of neuropathic pain.

In another aspect of the present invention, a method of identifying a compound useful for the treatment or prevention of neuropathic pain is provided, which comprises: (a) contacting a microglia in inactive-form with a test compound, in the presence of microglia-activator, (b) determining whether or not said test compound inhibits an activation of said microglia, and (c) identifying the test compound which inhibits said activation, as useful for the treatment or prevention of neuropathic pain.

In yet another aspect of the present invention, a therapeutic agent for treatment or prevention of neuropathic pain comprising P2X₄ receptor inhibitor is provided.

In yet another aspect of the present invention, a therapeutic agent for treatment or prevention of neuropathic pain comprising microglial activation-inhibitor is provided.

In yet another aspect of the present invention, a method for treating or preventing neuropathic pain comprising administering to a subject a therapeutically effective amount of P2X₄ receptor inhibitor is provided.

In yet another aspect of the present invention, a method for treating or preventing neuropathic pain comprising administering to a subject a therapeutically effective amount of microglial activation-inhibitor is provided.

The present invention is advantageous in enabling treatment and prevention of neuropathic pain, in particular, in enabling treatment of established condition of neuropathic pain.

### BRIEF DESCRIPTION OF DRAWINGS

FIG. 1 shows that spinal administration of TNP-ATP (2′,3′-O-(2,4,6-Trinitrophenyl) Adenosine 5′-Triphosphate) but not PPADS reverses tactile allodynia caused by L5 spinal nerve injury. The withdrawal threshold of tactile stimulation to the ipsilateral hindpaw was examined by applying the von Frey filaments. Panels (a) and (c) show paw withdrawal threshold (mean±SEM) prior to nerve injury (BL), 7 (a; Day 7) and 14 days (c; Day 14) after nerve injury (***P<0.001 vs. BL). The line graphs show the effects of intrathecal administration of TNP-ATP (30 nmol) and PPADS (30 nmol) on the decrease in paw withdrawal threshold 7 (a) and 14 days (c) after nerve injury (##P<0.01 and ###P<0.001 vs. PBS-treated group). Panels (b) and (d) show anti-allodynic effect (mean±SEM) of TNP-ATP 7 (b) and 14 days (d) after nerve injury (##P<0.01 and ###P<0.001 vs. PBS-treated group). Anti-allodynic effect (%)=100×(test value−pre-injection value)/(15.1 g−pre-injection value).

FIG. 2 shows dramatic upregulation of P2X₄R level in the spinal dorsal horn after L5 nerve injury. The upper panel shows western blot analysis of P2X₄R protein detected by P2X₄R antibody in the membrane fraction from the spinal cord ipsilateral to the nerve injury at different time. The total protein loaded on each lane was stained by Coomassie blue (middle). The time-course change in the P2X₄R protein is similar to that in paw withdrawal threshold (lower) [**P<0.01 and ***P<0.001 vs. pre-injury baseline (BL)].

FIG. 3 shows that P2X₄R is induced in hyperactive microglia. All experiments were done using the spinal cord sections 14 days after nerve injury. Panel (a) shows the number of cells labelled with OX42 (mean±SEM/10⁴ μm²) in the dorsal horn ipsilateral (Ipsi) and contralateral (Contra) to the nerve injury (***P<0.001 vs. contralateral). Panel (b) shows Immunofluorescence (IF) intensity of P2X₄R protein in individual microglia determined as the average pixel density in the ipsilateral (Ipsi; n=236 OX42-positive cells) and contralateral (Contra; n=104 OX42-positive cells) dorsal horn (***P<0.001). Data are shown by the percentage (mean±SEM) of the values normalised to the mean values of the contralateral dorsal horn. Panel (c) shows a histogram of the percentage of dorsal horn microglia displaying ranges of immunofluorescence (IF) intensity values of P2X₄R protein in individual microglia.

FIG. 4 shows that P2X₄R antisense oligodeoxynucleotide (ODN) suppresses the development of tactile allodynia caused by L5 spinal nerve injury. Rats were injected intrathecally with antisense ODN (5 nmol) or mismatch ODN (5 nmol) once a day for 7 days. Panel (a) shows paw withdrawal threshold (mean±SEM) of tactile stimulation to the hindpaw ipsilateral to the nerve injury. BL—baseline prior to nerve injury; MM—animals (n=10) treated with mismatch ODN; AS—animals (n=11) treated with antisense ODN (**P<0.01). Panel (b) shows Western blot and immunocytochemical analyses of P2X₄R protein in the spinal cord and in individual microglia in the dorsal horn, respectively, of antisense ODN (AS)— and mismatch ODN (MM)-treated rats. Data obtained from antisense and mismatch ODN-treated animals were quantified and normalised to the values of mismatch ODN-treated group (mean±SEM). The behavioural effect of antisense ODN was converted into the percentage of tactile allodynia which was calculated by the formula: tactile allodynia (%)=100×(15.1 g−value of antisense ODN)/(15.1 g−value of mismatch ODN) (**P<0.01). IB—immunoblot in homogenates from the spinal cord (**P<0.01); IF—immunofluorescence intensity in individual microglia (***P<0.001). Panel (c) shows the number of microglia and intensity of OX42 immunofluorescence in individual microglia in the dorsal spinal cord of antisense ODN (AS)- and mismatch ODN (MM)-treated rats. Data were quantified and normalised to the values of mismatch ODN-treated group (mean±SEM).

FIG. 5 shows that spinal administration of ATP-stimulated microglia in normal rats produces tactile allodynia that depends upon P2X₄Rs. Panel (a) shows diagram illustrating the experimental protocol. Panel (b) shows paw withdrawal threshold (mean±SEM) of tactile stimulation after intrathecal administration of PBS, ATP (50 μM) and microglia that preincubated with PBS or ATP (50 μM) for 1 hr (***P<0.001 vs. PBS-treated microglia group). TNP-ATP (10 μM) was pretreated with microglia 10 min prior to ATP application (###P<0.001 vs. ATP-stimulated microglia-treated group). Panel (c) shows reversal by intrathecal administration of TNP-ATP (30 nmol) 5 hr after the microglia injection of tactile allodynia caused by the ATP-stimulated microglia (**P<0.01 vs. pre-injection baseline, #P<0.05 and ##P<0.01 vs. the value at 5 hr after the microglia injection, +P<0.05 vs. the value at 75 min after the injection of TNP-ATP). Data are shown by the mean±SEM of paw withdrawal threshold (in grams).

FIG. 6 shows that functional P2X₄R is expressed in hyperactive microglia in primary culture. Panels (a) and (b) show intracellular Ca 2+([Ca²⁺]i) imaging analysis of individual microglia using the Ca²⁺-sensitive fluorescent dye fura-2. The traces show ATP (50 μM, 10 s)-evoked a transient increase in the 340/360 emission ratio for fura-2 in microglia under the conditions with or without adding Ca²⁺ in the extracellular solution. The graphs show relative increase ratio (Δ340/F360; mean±SEM) from the basal level before ATP application (***P<0.001, n=28 cells) (a). ATP (50 μM)-evoked a transient increase in the 340/360 emission ratio (Δ340/F360; mean±SEM) is suppressed by pretreatment with 10 μM TNP-ATP (**P<0.01, n=14 cells), but not with 10 μM PPADS (n=26 cells) and with 100 nM brilliant blue G (BBG, n=21 cells), a selective antagonist for P2X₇R (Jiang, L. H., Mackenzie, A. B., North, R. A. & Surprenant, A. Brilliant blue G selectively blocks ATP-gated rat P2X₇ receptors. Mol Pharmacol 58, 82-88, 2000) (b).

### BRIEF DESCRIPTION OF SEQUENCES

SEQ ID NO: 1 represents a coding sequence of human P2X₄ receptor gene.

SEQ ID NO: 2 represents an amino acid sequence of human P2X₄ receptor.

SEQ ID NO: 3 represents a nucleotide sequence of antisense oligonucleotide for suppressing expression of human P2X₄ receptor gene.

SEQ ID NO: 4 represents a nucleotide sequence of mismatched antisense oligonucleotide for use as negative control of antisense oligonucleotide depicted in SEQ ID NO: 3.

SEQ ID NO: 5 and 6 respectively represent forward and reverse primers for amplification of rat P2X₁ receptor gene.

SEQ ID NO: 7 and 8 respectively represent forward and reverse primers for amplification of rat P2X₂ receptor gene.

SEQ ID NO: 9 and 10 respectively represent forward and reverse primers for amplification of rat P2X₃ receptor gene.

SEQ ID NO: 11 and 12 respectively represent forward and reverse primers for amplification of rat P2X₄ receptor gene.

SEQ ID NO: 13 and 14 respectively represent forward and reverse primers for amplification of rat P2X₅ receptor gene.

SEQ ID NO: 15 and 16 respectively represent forward and reverse primers for amplification of rat P2X₆ receptor gene.

SEQ ID NO: 17 and 18 respectively represent forward and reverse primers for amplification of rat P2X₇ receptor gene.

SEQ ID NO: 19 represents a coding sequence of human P2Y₁₂ receptor gene.

SEQ ID NO: 20 represents an amino acid sequence of human P2Y₁₂ receptor.

### DETAILED DESCRIPTION OF THE INVENTION

In the present study we demonstrate that activation of P2X₄Rs induced in spinal cord microglia is essential for tactile allodynia following peripheral nerve injury. Tactile allodynia was reversed rapidly by pharmacological blockade of these receptors implying that nerve injury-induced pain hypersensitivity depends upon ongoing signalling via P2X₄Rs, likely activated by ATP which may be released from primary sensory terminals (Sawynok, J., Downie, J. W., Reid, A. R., Cahill, C. M. & White, T. D. ATP release from dorsal spinal cord synaptosomes: characterization and neuronal origin. Brain Res 610, 32-38. (1993); Li, P., Calejesan, A. A. & Zhuo, M. ATP P2X receptors and sensory synaptic transmission between primary afferent fibers and spinal dorsal horn neurons in rats. J Neurophysiol 80, 3356-3360. (1998); Nakatsuka, T. & Gu, J. G. ATP P2X receptor-mediated enhancement of glutamate release and evoked EPSCs in dorsal horn neurons of the rat spinal cord. J Neurosci 21, 6522-6531. (2001)), dorsal horn neurons (Sawynok, J., Downie, J. W., Reid, A. R., Cahill, C. M. & White, T. D. ATP release from dorsal spinal cord synaptosomes: characterization and neuronal origin. Brain Res 610, 32-38. (1993); Bardoni, R., Goldstein, P. A., Lee, C. J., Gu, J. G. & MacDermott, A. B. ATP P2X receptors mediate fast synaptic transmission in the dorsal horn of the rat spinal cord. J Neurosci 17, 5297-304. (1997); Jo, Y. H. & Schlichter, R. Synaptic corelease of ATP and GABA in cultured spinal neurons. Nat Neurosci 2, 241-245. (1999)), or from dorsal horn astrocytes (Fam, S. R., Gallagher, C. J. & Salter, M. W. P2Y₁ purinoceptor-mediated Ca²⁺ signaling and Ca²⁺ wave propagation in dorsal spinal cord astrocytes. J Neurosci 20, 2800-2808. (2000)) (see also Supplementary Information 2). As a consequence of peripheral nerve injury microglia in the spinal dorsal horn are converted to the hyperactive phenotype and have dramatically increased expression of P2X₄Rs. As we show that in hyperactive microglia activating P2X₄Rs provokes entry of Ca²⁺ and that administering P2X₄R-stimulated hyperactive microglia produces tactile allodynia in normal rats, pain hypersensitivity may result from release from these microglia of bioactive factors, such as cytokines (Inoue, K. Microglial activation by purines and pyrimidines. Glia 40, 156-163. (2002); Hanisch, U. K. Microglia as a source and target of cytokines. Glia 40, 140-155. (2002); Vitkovic, L., Bockaert, J. & Jacque, C. “Inflammatory” cytokines: neuromodulators in normal brain? J Neurochem 74, 457-471. (2000)), that enhance synaptic transmission in spinal pain pathways. Thus, preventing the upregulation of P2X₄R expression in spinal microglia or inhibiting these receptors pharmacologically represent novel therapeutic approaches for treating pain hypersensitivity caused by nerve damage, for which there is currently no effective therapy. As pharmacological blockade of these receptors did not affect pain responses in naive animals, a predicted therapeutic benefit of interfering with P2X₄Rs is that normal pain sensitivity would be unaffected. In this study we have discovered a critical role of hyperactive microglia in a model of neuropathic pain, a common pathological condition of the CNS. Hyperactive microglia are considered crucial for the pathogenesis of various other pathological conditions of the CNS, such as neurodegenerative disorders and stroke (Nakajima, K. & Kohsaka, S. Functional roles of microglia in the brain. Neurosci Res 17, 187-203. (1993); Carson, M. J. Microglia as liaisons between the immune and central nervous systems: Functional implications for multiple sclerosis. Glia 40, 218-231. (2002); Eikelenboom, P. et al. Neuroinflammation in Alzheimer's disease and prion disease. Glia 40, 232-239. (2002)). If P2X₄Rs are increased in microglia in these conditions then they may represent a novel therapeutic target in CNS disorders in addition to nerve injury-induced pain hypersensitivity.

Accordingly, the present invention provides a method of identifying a compound useful for the treatment or prevention of neuropathic pain. This method can be accomplished by identifying a P2X₄ receptor inhibitor, i.e. a compound which inhibits the action of P2X₄ receptor, using standard techniques well-known in the art. The method thus comprises the following steps: (a) contacting a cell expressing P2X₄ receptor on the surface thereof, with a test compound, in the presence of P2X₄ receptor agonist, (b) determining whether or not said test compound inhibits an interaction of said P2X₄ receptor agonist and P2X₄ receptor on the surface of the cell, and (c) identifying the test compound which inhibits said interaction, as useful for the treatment or prevention of neuropathic pain (this method is hereinafter referred to as “the identification method 1”).

The term “neuropathic pain” as used herein means pain induced by expression of pathological operation of the nervous system following nerve injury due to various causes, for example, surgical operation, wound, shingles, diabetic neuropathy, amputation of legs or arms, cancer, and the like. The neuropathic pain is preferably tactile allodynia induced after nerve injury.

The cell employed in the identification method 1 may be those which naturally express the P2X₄ receptor on the surface thereof. Alternatively, the cell may also be transfected to express the receptor on the surface thereof. An amino acid sequence of human P2X₄ receptor and a nucleotide sequence encoding it are exemplified in SEQ ID NOS: 2 and 1 (NCBI Accession No: AF191093), respectively. Referring to these sequences, the cell can be appropriately transfected according to standard procedures well-known in the art, for example, as described in Sambrook et al., MOLECULAR CLONING, A LABORATORY MANUAL, 2nd Ed.; Cold Spring Harbor Laboratory Press, Cold Spring Harbor, N.Y. (1989).

In a preferred embodiment, a mammalian cell is employed in the identification method 1 according to the present invention. Various mammalian cell culture systems can be employed to express P2X₄ receptor on the surface of the cell. Examples of mammalian cells include COS-7, C127, 3T3, CHO, HEK293, HeLa and BHK cell lines. Expression vectors for use in mammalian cells will comprise, for example, an origin of replication, a suitable promoter and enhancer, and also any necessary ribosome binding sites, polyadenylation site, splice donor and acceptor sites, transcriptional termination sequences, and 5′ UTR.

In another preferred embodiment, the cell employed in the identification method 1 according to the present invention does not express any P2X receptors other than P2X₄ receptor, such as P2X₁, P2X₂, P2X₃, P2X₅, P2X₆, and P2X₇ receptors. Such cells can be easily obtained or prepared by those skilled in the art. For example, such cells can be prepared by introducing P2X₄ receptor-expression vector into cells which does not naturally express any P2X receptors, or by knocking out or knocking down all of the P2X receptor genes other than P2X₄ receptor gene in cells which naturally express P2X₄ receptor. These engineering can be carried out according to standard procedures well-known in the art, for example, transfection techniques or gene silencing techniques.

The P2X₄ receptor agonist is well-known in the art, including synthetic compounds and naturally occurring agonists. The naturally occurring agonist typically includes ATP and ADP.

The contacting step (a) in the identification method 1 can be carried out by standard procedures well-known in the art, for example, by incubating the cell and the test compound in the presence of the P2X₄ receptor agonist. In a preferred embodiment of the present invention, the step (a) comprises incubating the cell and the test compound in the absence of the P2X₄ receptor agonist, and then incubating them in the presence of the P2X₄ receptor agonist. Those skilled in the art will be able to select medium, temperature, time length, amount of the test compound and the cell, and preferred or required additives, useful for the incubation, depending on the test compound and the cell.

In the determining step (b) in the identification method 1, the inhibitory activity of a test compound can be determined by standard techniques well-known in the art, for example, by measuring intensity of the interaction of the P2X₄ receptor agonist and P2X₄ receptor. P2X₄ receptor is known to form the ion-channel for Na⁺, K⁺, and Ca²⁺. Therefore, the interaction can be determined by measuring P2X₄ receptor-mediated ion flux of at least one ion selected from the group consisting of Na⁺, K⁺, and Ca²⁺ by standard procedures well-known in the art. For this purpose, it is preferred that the contacting step (a) is carried out in the presence of the ion. For example, the flux of the ion can be determined by measuring content of the ion in nitric acid-extracts of the cell by atomic absorbance spectrophotometry. In a preferred embodiment of the present invention, the step (b) comprises comparing intensity of the interaction with that of control sample obtained in the absence of any test compounds. The control sample can be prepared by carrying out the step (a) in the same way, provided that no test compound is added.

In a preferred embodiment of the present invention, a selective P2X₄ receptor inhibitor is identified as useful for the treatment or prevention of neuropathic pain. The term “selective” as used for the P2X₄ receptor inhibitor means that the P2X₄ receptor inhibitor inhibits the action of P2X₄ receptor, whereas it does not substantially inhibit the action of all the other P2X receptors, such as P2X₁, P2X₂, P2X₃, P2X₅, P2X₆, and P2X₇ receptors. More preferably, the selective P2X₄ receptor inhibitor also does not substantially inhibit the action of all of the P2Y receptors, such as P2Y₁, P2Y₂, P2Y₄, P2Y₆, P2Y₁₁, P2Y₁₂, and P2Y₁₃ receptors. Such a selective P2X₄ receptor inhibitor can be identified by confirming that the test compound has inhibitory activity on P2X₄ receptor according to the above method, and that a test compound has substantially no inhibitory activity on any P2X receptors other than P2X₄ receptor in the same way as the above method. Further, the test compound can be confirmed not to substantially inhibit the action of all of the P2Y receptors by well-known procedure for G protein-coupled receptor, for example, by measuring calcium mobilization, ⁴⁵Ca efflux or measurements of intracellular Ca²⁺ concentration with fluorescent dyes such as fura-2 and voltage clamp (for example, see WO 01/46454). The phrases “does not substantially inhibit” and “has substantially no inhibitory activity” as used herein mean that the compound has much lower inhibitory activity on a certain P2 receptor (excluding P2X₄ receptor) than that on P2X₄ receptor. The inhibitory activity of the compound on a certain P2 receptor (excluding P2X₄ receptor) is, for example, but not limited to, 1/10 fold, preferably 1/100 fold, more preferably 1/1000 fold lower than that on P2X₄ receptor. The inhibitory activity of the compound on a certain P2Y receptor may be compared with that on P2X₄ receptor by well-known binding assay. The value of the inhibitory activity for use in the above comparison may be an amount of the compound required for predetermined degree of inhibition.

In addition, the treatment or prevention of neuropathic pain can also be accomplished by inhibiting activation of microglia in spinal cord to suppress the expression of P2X₄ receptors in the microglia. Therefore, in another aspect, the present invention provides a method of identifying a compound useful for the treatment or prevention of neuropathic pain by identifying a microglial activation-inhibitor, i.e. a compound which inhibits activation of microglia, using standard techniques well-known in the art. The method thus comprises the following steps: (a) contacting a microglia in inactive-form with a test compound, in the presence of microglia-activator, (b) determining whether or not said test compound inhibits an activation of said microglia, and (c) identifying the test compound which inhibits said activation, as useful for the treatment or prevention of neuropathic pain (this method is hereinafter referred to as “the identification method 2”).

The microglia can be obtained by those skilled in the art, for example, from central nerve system. The microglia is known to be either in active-form (amoeboid form) or in inactive-form (ramified form). Further, the microglia in active-form is known to have a ruffling structure. Therefore, the microglia in inactive-form employed in the identification method 2 can be easily selected by those skilled in the art, for example, according to the form of the microglia such as absence of the ruffling formation.

The microglia-activator is well known in the art, including synthetic compounds and naturally occurring activators, preferably naturally occurring activators such as ATP and ADP.

The contacting step (a) in the identification method 2 can be carried out by standard procedures well-known in the art, for example, by incubating the microglia in inactive-form and the test compound in the presence of the microglia-activator. In a preferred embodiment of the present invention, the step (a) comprises incubating the microglia and the test compound in the absence of the microglia-activator, and then incubating them in the presence of the microglia-activator. Those skilled in the art will be able to select medium, temperature, time length, amount of the test compound and the microglia, and preferred or required additives, useful for the incubation.

In the determining step (b) in the identification method 2, the inhibitory activity of a test compound can be determined by standard techniques well-known in the art, for example, by measuring intensity of the activation of the microglia. As described above, the microglia in active-form exhibits amoeboid form with ruffling formation, and the microglia in inactive-form exhibits ramified form. Therefore, the microglia in active-form and inactive-form can be easily selected from their mixture by those skilled in the art according to their forms. In a preferred embodiment of the present invention, the step (b) comprises comparing intensity of the activation with that of control sample obtained in the absence of any test compounds. The control sample can be prepared by carrying out the step (a) in the same way, provided that no test compound is added.

Further, the activation of the microglia by ATP or ADP is known to be caused by the action of P2Y₁₂ receptor. Therefore, P2Y₁₂ receptor inhibitor is useful as the microglial activation-inhibitor, i.e. useful for the treatment or prevention of neuropathic pain. Accordingly, in yet another aspect, the present invention provides a method of identifying a compound useful for the treatment or prevention of neuropathic pain by identifying a P2Y₁₂ receptor inhibitor, i.e. a compound which inhibits the action of P2Y₁₂ receptor, using standard techniques well-known in the art. The method thus comprises the following steps: (a) contacting a cell expressing P2Y₁₂ receptor on the surface thereof, with a test compound, in the presence of P2Y₁₂ receptor agonist, (b) determining whether or not said test compound inhibits an interaction of said P2Y₁₂ receptor agonist and P2Y₁₂ receptor on the surface of the cell, and (c) identifying the test compound which inhibits said interaction, as useful for the treatment or prevention of neuropathic pain (this method is hereinafter referred to as “the identification method 3”).

The cell employed in the identification method 3 may be those which naturally express the P2Y₁₂ receptor on the surface thereof. Alternatively, the cell may also be transfected to express the receptor on the surface thereof. An amino acid sequence of human P2Y₁₂ receptor and a nucleotide sequence encoding it are exemplified in SEQ ID NOS: 20 and 19 (NCBI Accession Nos: NM022788 and NM176876), respectively. Referring to these sequences, the cell can be appropriately transfected according to standard procedures well-known in the art, for example, as described in Sambrook et al., MOLECULAR CLONING, A LABORATORY MANUAL, 2nd Ed.; Cold Spring Harbor Laboratory Press, Cold Spring Harbor, N.Y. (1989).

In a preferred embodiment, a mammalian cell is employed in the identification method 3 according to the present invention. Various mammalian cell culture systems can be employed to express P2Y₁₂ receptor on the surface of the cell. Examples of mammalian cells include COS-7, C127, 3T3, CHO, HEK293, HeLa and BHK cell lines. Expression vectors for use in mammalian cells will comprise, for example, an origin of replication, a suitable promoter and enhancer, and also any necessary ribosome binding sites, polyadenylation site, splice donor and acceptor sites, transcriptional termination sequences, and 5′ UTR.

In another preferred embodiment, the cell employed in the identification method 3 according to the present invention does not express any P2Y receptors other than P2Y₁₂ receptor, such as P2Y₁, P2Y₂, P2Y₄, P2Y₆, P2Y₁₁, and P2Y₁₃ receptors. Such cells can be easily obtained or prepared by those skilled in the art. For example, such cells can be prepared by introducing P2Y₁₂ receptor-expression vector into cells which does not naturally express any P2Y receptors, or by knocking out or knocking down all of the P2Y receptor genes other than P2Y₁₂ receptor gene in cells which naturally express P2Y₁₂ receptor. These engineering can be carried out according to standard procedures well-known in the art, for example, transfection techniques or gene silencing techniques.

In the cell employed in the identification method 3, P2Y₁₂ receptor is preferably expressed as Gi protein-coupled receptor by well-known techniques including, for example, those disclosed in WO 01/46454.

The P2Y₁₂ receptor agonist is well-known in the art, including synthetic compounds and naturally occurring agonists. The naturally occurring agonist typically includes ATP and ADP.

The contacting step (a) in the identification method 3 can be carried out by standard procedures well-known in the art, for example, by incubating the cell and the test compound in the presence of the P2Y₁₂ receptor agonist. In a preferred embodiment of the present invention, the step (a) comprises incubating the cell and the test compound in the absence of the P2Y₁₂ receptor agonist, and then incubating them in the presence of the P2Y₁₂ receptor agonist. Those skilled in the art will be able to select medium, temperature, time length, amount of the test compound and the cell, and preferred or required additives, useful for the incubation, depending on the test compound and the cell.

In the determining step (b) in the identification method 3, the inhibitory activity of a test compound can be determined by standard techniques well-known in the art, for example, by measuring intensity of the interaction of the P2Y₁₂ receptor agonist and P2Y₁₂ receptor. P2Y₁₂ receptor is known to form the Gi protein-coupled receptor with the Gi protein. Therefore, the interaction can be determined by measuring calcium mobilization, ⁴⁵Ca efflux or measurements of intracellular Ca²⁺ concentration with fluorescent dyes such as fura-2 and voltage clamp. These measurements are carried out by standard techniques well-known in the art including, for example, those disclosed in WO 01/46454. In a preferred embodiment of the present invention, the step (b) comprises comparing intensity of the interaction with that of control sample obtained in the absence of any test compounds. The control sample can be prepared by carrying out the step (a) in the same way, provided that no test compound is added.

In a preferred embodiment of the present invention, a selective P2Y₁₂ receptor inhibitor is identified as useful for the treatment or prevention of neuropathic pain. The term “selective” as used for the P2Y₁₂ receptor inhibitor means that the P2Y₁₂ receptor inhibitor inhibits the action of P2Y₁₂ receptor, whereas it does not substantially inhibit the action of all the other P2Y receptors, such as P2Y₁, P2Y₂, P2Y₄, P2Y₆, P2Y₁₁, and P2Y₁₃ receptors. More preferably, the selective P2Y₁₂ receptor inhibitor also does not substantially inhibit the action of all of the P2X receptors, such as P2X₁, P2X₂, P2X₃, P2X₄, P2X₅, P2X₆, and P2X₇ receptors. Such a selective P2Y₁₂ receptor inhibitor can be identified by confirming that the test compound has inhibitory activity on P2Y₁₂ receptor according to the above method, and that a test compound has substantially no inhibitory activity on any P2Y receptors other than P2Y₁₂ receptor in the same way as the above method. Further, the test compound can be confirmed not to substantially inhibit the action of all of the P2X receptors by well-known procedure for ion-channel receptor, such as those exemplified above. The phrases “does not substantially inhibit” and “has substantially no inhibitory activity” as used herein mean that the compound has much lower inhibitory activity on a certain P2 receptor (excluding P2Y₁₂ receptor) than that on P2Y₁₂ receptor. The inhibitory activity of the compound on a certain P2 receptor (excluding P2Y₁₂ receptor) is, for example, but not limited to, 1/10 fold, preferably 1/100 fold, more preferably 1/1000 fold lower than that on P2Y₁₂ receptor. The inhibitory activity of the compound on a certain P2X receptor may be compared with that on P2Y₁₂ receptor by well-known binding assay. The value of the inhibitory activity for use in the above comparison may be an amount of the compound required for predetermined degree of inhibition.

The P2X₄ receptor inhibitor or the microglial activation-inhibitor is useful for the treatment or prevention of neuropathic pain, such as tactile allodynia induced after nerve injury. Therefore, the present invention further provides a use of P2X₄ receptor inhibitor or microglial activation-inhibitor for treating or preventing neuropathic pain. In addition, the present invention also provides a method for treating or preventing neuropathic pain comprising administering to a subject a therapeutically effective amount of P2X₄ receptor inhibitor or microglial activation-inhibitor. Further, the present invention also provides a use of P2X₄ receptor inhibitor or microglial activation-inhibitor for manufacture of medicament for treatment or prevention of neuropathic pain.

The P2X₄ receptor inhibitor can be identified by the identification method 1 according to the present invention. The microglial activation-inhibitor can be identified by the identification method 2 according to the present invention. The microglial activation-inhibitor is preferably a P2Y₁₂ receptor inhibitor which can be identified by the identification method 3 according to the present invention.

In one embodiment of the present invention, the P2X₄ receptor inhibitor and the P2Y₁₂ receptor inhibitor are a P2X₄ receptor antagonist and a P2Y₁₂ receptor antagonist, respectively. The term “P2X₄ receptor antagonist” as used herein means a small molecule which binds to the P2X₄ receptor, and makes it inaccessible to its agonist, such as ATP and ADP, such that the action of the P2X₄ receptor is prevented. Similarly, the term “P2Y₁₂ receptor antagonist” as used herein means a small molecule which binds to the P2Y₁₂ receptor, and makes it inaccessible to its agonist, such as ATP and ADP, such that the action of the P2Y₁₂ receptor is prevented. Examples of such a small molecules include, but not limited to, small peptides or peptide-like molecules, organic molecules such as TNP-ATP (2′,3′-O-(2,4,6-Trinitrophenyl) Adenosine 5′-Triphosphate) for P2X₄ receptor or AR-C69931 MX (N⁶-(2-methylthioethyl)-2-(3,3,3-trifluoropropylthio)-β,γ-dichloromethylene ATP) for P2Y₁₂ receptor, and the like.

In another embodiment of the present invention, the P2X₄ receptor inhibitor or the P2Y₁₂ receptor inhibitor is an antibody or an antibody fragment which binds to the receptor protein on the surface of the cell and prevents the interaction between the receptor and its agonist, such as ATP and ADP. Such an antibody and an antibody fragment typically bind to a soluble extracellular fragment of the receptor protein. Such an extracellular fragment can be easily designed by those skilled in the art, based on an amino acid sequence of P2X₄ receptor, such as SEQ ID NO: 2, or an amino acid sequence of P2Y₁₂ receptor, such as SEQ ID NO: 20. The antibody may be either polyclonal or monoclonal, and may be either intact form or functional fragment thereof such as Fab′, F(ab′)₂, and the like. These antibodies and fragments thereof can be designed and prepared according to standard procedures well-known in the art.

In yet another embodiment of the present invention, the P2X₄ receptor inhibitor or the P2Y₁₂ receptor inhibitor is an antisense nucleic acid molecule that specifically suppresses expression of corresponding receptor gene. Antisense technology is well-known technique to suppress gene expression. In one exemplary technique, the antisense nucleic acid molecule is an antisense RNA oligonucleotide of from about 10 to 40 bases in length designed based on the 5′ coding portion of the receptor gene. In another exemplary technique, the antisense nucleic acid molecule is a DNA oligonucleotide designed to be complementary to a region of the receptor gene involved in its transcription. Such an antisense nucleic acid molecule can be easily designed by those skilled in the art, based on a specific nucleotide sequence for the receptor gene such as SEQ ID NO: 1 for P2X₄ receptor or SEQ ID NO: 19 for P2Y₁₂ receptor.

In yet another embodiment of the present invention, the P2X₄ receptor inhibitor or the P2Y₁₂ receptor inhibitor is an siRNA nucleic acid molecule that specifically suppresses expression of corresponding receptor gene. The term “siRNA nucleic acid molecule” as used herein means not only siRNA per se, but also longer double-strand RNA molecule which can provide siRNA in target cells. The siRNA nucleic acid molecule can suppress gene expression through RNAi, and is a well-know powerful tool for gene silencing (Elbashir, S. M. et al., Nature 411, 494-498, 2001). The siRNA typically comprises a nucleotide sequence of 19 to 21 base pairs homologous to a specific sequence for mRNA of the receptor gene. The longer double-strand RNA molecule typically comprises a longer nucleotide sequence homologous to a specific sequence for mRNA of the receptor gene. Such an siRNA nucleic acid molecule can be easily designed by those skilled in the art, based on a specific nucleotide sequence for the receptor gene such as SEQ ID NO: 1 for P2X₄ receptor or SEQ ID NO: 19 for P2Y₁₂ receptor. The siRNA nucleic acid molecule can also be expressed from a suitable vector delivered to cells. Accordingly, the P2X₄ receptor inhibitor or the P2Y₁₂ receptor inhibitor may be a vector expressing the siRNA nucleic acid molecule that specifically suppresses expression of corresponding receptor gene. Such a vector can be easily constructed by standard procedures well-known in the art (Bass, B. L., Cell 101, 235-238, 2000; Tavernarakis, N. et al., Nat. Genet. 24, 180-183, 2000; Malagon, F. et al., Mol. Gen. Genet. 259, 639-644, 1998; Parrish, S. et al., Mol. Cell 6, 1077-1087, 2000).

In a preferred embodiment, a selective P2X₄ receptor inhibitor or a selective P2Y₁₂ receptor inhibitor is employed in the therapeutic agent and the therapeutic method according to the present invention. The selective receptor inhibitor is advantageous in enabling prevention of potential side effects which are not desired for a subject. The selective P2X₄ receptor inhibitor and the selective P2Y₁₂ receptor inhibitor can be identified as described above.

The P2X₄ receptor inhibitor or the microglial activation-inhibitor may be administered in a convenient manner enabling delivery to spinal microglia, preferably microglia in ipsilateral spinal cord with a site of nerve injury. Accordingly, the P2X₄ receptor inhibitor or the microglial activation-inhibitor is preferably administered intraspinally, more preferably by intrathecal injection. Therapeutically effective amount of the P2X₄ receptor inhibitor or the microglial activation-inhibitor can be determined by a practitioner, depending on the severity of condition, the age and species of the subject, inhibitory activity of the particular inhibitor, the route, timing, and frequency of administration, and the like. In general, the P2X₄ receptor inhibitor or the microglial activation-inhibitor will be administered in an amount of about 0.001 to about 1000 mg/kg body weight per day, preferably about 0.01 to about 10 mg/kg body weight per day, more preferably about 0.01 to about 1 mg/kg body weight per day. The subject is preferably mammal, for example, human or non-human mammal.

The P2X₄ receptor inhibitor or the microglial activation-inhibitor may be administered in admixture with a pharmaceutically acceptable carrier. Accordingly, the present invention further provides a pharmaceutical composition comprising the P2X₄ receptor inhibitor or the microglial activation-inhibitor, and a pharmaceutically acceptable carrier. Such a pharmaceutical composition can be used for treatment or prevention of neuropathic pain. The pharmaceutically acceptable carrier, such as vehicles, excipients, diluents, and the like, can be selected by those skilled in the art, depending on the route of administration. Preferably, the pharmaceutical composition according to the present invention comprises a therapeutically effective amount of P2X₄ receptor inhibitor or microglial activation-inhibitor, and a pharmaceutically acceptable carrier.

The present invention further provides a commercial package comprising the pharmaceutical composition according to the present invention, and a written matter which states that the pharmaceutical composition can or should be used for treatment or prevention of neuropathic pain.

It should be understood that all contents of the documents cited above and below are incorporated herein by reference.

The following examples further illustrate the present invention. The examples should not be construed as in any way limiting the scope of the present invention.

### EXAMPLE

### Example 1

### Mechanisms Underlying Tactile Allodynia after Nerve Injury

Methods

Behavioural Studies

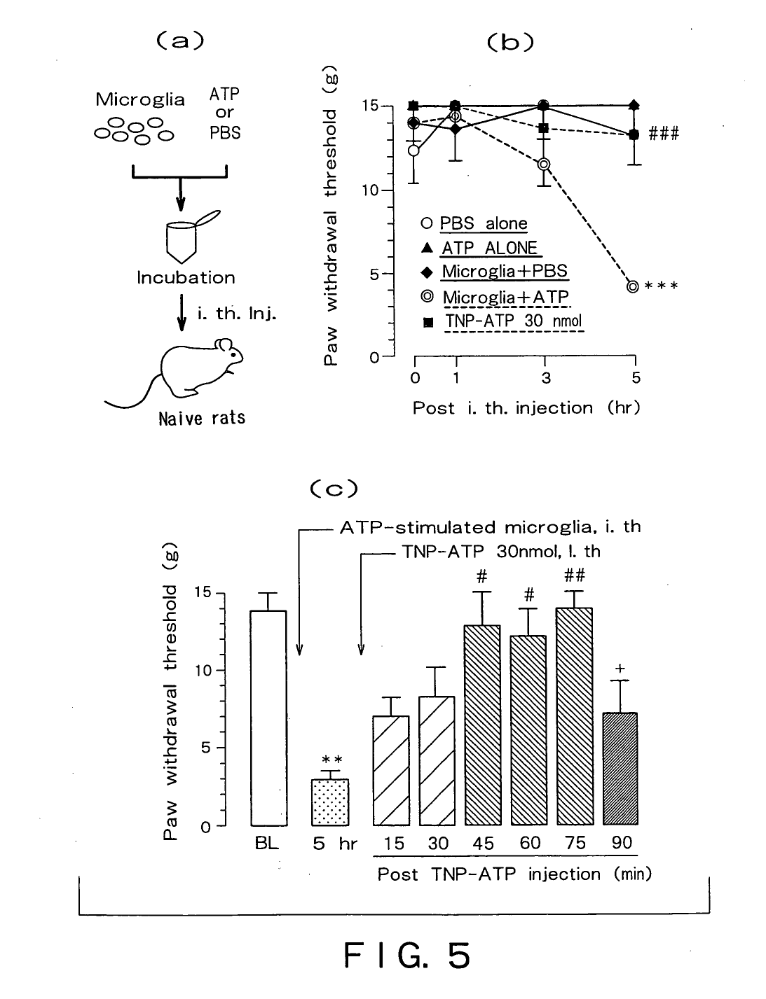

We used the spinal nerve injury model (Kim, S. H. & Chung, J. M. An experimental model for peripheral neuropathy produced by segmental spinal nerve ligation in the rat. Pain 50, 355-363. (1992)) with some modifications; a unilateral L5 spinal nerve of male Wistar rats was tightly ligated and cut just distal to the ligature. The mechanical allodynia was assessed in naive and nerve-injured rats using calibrated von Frey filaments (0.4-15.1 g). Rats were injected intrathecally with P2XR antagonists, TNP-ATP and PPADS (Sigma-RBI), and antisense and mismatch ODN. For the experiments in FIG. 5, the cultured microglia (see Supplementary Information 1) that had been pre-incubated with or without ATP (50 μM) were injected intrathecally in normal rats. Full details of experimental methods are as follows.

A unilateral L5 spinal nerve of male Wistar rats was tightly ligated and cut just distal to the ligature. To assess the mechanical allodynia, the calibrated von Frey filaments (0.4-15.1 g) were applied to the plantar surface of the hindpaw from below the mesh floor. The 50% paw withdrawal threshold was determined using the up-down method (Chaplan, S. R., Bach, F. W., Pogrel, J. W., Chung, J. M. & Yaksh, T. L. Quantitative assessment of tactile allodynia in the rat paw. J Neurosci Methods 53, 55-63, 1994). Rats were implanted with a PE-10 polyethylene tube to the lumber enlargement (Yaksh, T. L., Jessell, T. M., Gamse, R., Mudge, A. W. & Leeman, S. E. Intrathecal morphine inhibits substance P release from mammalian spinal cord in vivo. Nature 286, 155-157, 1980) for intrathecal injection of P2XR antagonists [2′,3′-O-(2,4,6-trinitrophenyl)adenosine 5′-triphosphate (TNP-ATP) (Sigma-RBI) and pyridoxalphosphate-6-azophenyl-2′,4′-disulphonic acid (PPADS) (Sigma-RBI)], P2X₄R antisense or mismatch ODN. We confirmed that PPADS and TNP-ATP used in the present study show biological activity to inhibit effects of α,β-methylene ATP in vitro and in vivo. The 16-base antisense ODN endcapped with phosphorothioate linkages were designed according to the primary sequence of the rat P2X₄R cDNA (X87763). The sequence of the P2X₄R antisense and mismatch ODN was 5′-CAGCCCGCCATGGCTC-3′ (SEQ ID NO: 3) and 5′-ACCGCCGCCAGTGCCT-3′ (SEQ ID NO: 4), respectively. The mismatch ODN served as a control. Rats were injected intrathecally with antisense ODN (5 nmol 10 μl⁻¹) and mismatch ODN (5 nmol 10 μl⁻¹) using a 25-μl Hamilton syringe with 28-gauge needle once a day from day 0 (immediately after nerve injury) to day 6. On day 7, paw withdrawal threshold of antisense ODN- and mismatch ODN-treated rats were tested. After the test, to quantify the levels of P2X₄R protein in the spinal cord in both groups using Western blot analysis, the membrane fraction from the ipsilateral spinal cord segments L4-L6 was prepared. For the experiments in which cultured microglia were administered intrathecally, the cultured microglia were pre-incubated with ATP (50 μM) or PBS for 1 hr at 37° C. immediately prior to administration. Microglia with their supernatant were injected intrathecally in normal rats and paw withdrawal threshold was tested 1, 3 and 5 hr later. TNP-ATP (10 μM) was preincubated with microglia starting 10 min prior to ATP application.

Immunohistochemistry

Transverse L5 spinal cord sections (30 μm) were cut and processed for immunohistochemistry using P2X₄R antibody (Alomone). Markers of microglia, OX42 (Chemicon) and iba1 (gifted from S. Kohsaka); astrocytes, GFAP (Boehringer Mannheim); spinal cord neurones, NeuN (Chemicon) and MAP2 (Chemicon) were used to identify the type of P2X₄R-positive cells. To assess immunofluorescence staining of cells quantitatively, we measured the immunofluorescence intensity of the P2X₄R or OX42 as the average pixel intensity within each cell. Full details of experimental methods are as follows.

Transverse L5 spinal cord sections (30 μm) were incubated for 2 hr at room temperature in a blocking solution (3% normal goat serum) and then incubated for 48 hr at 4° C. in the primary antibody for P2X₄R (anti-P2X₄ receptor, 1:500, Alomone). Markers of microglia, OX42 (anti-OX42, 1:100, Chemicon) and iba1 (anti-iba1, 1:2000, gifted from S. Kohsaka); astrocytes, glial fibrillary acidic protein (GFAP, anti-GFAP, 1:500, Boehringer Mannheim); spinal cord neurones, neuronal marker (NeuN, anti-NeuN, 1:200, Chemicon) and microtubule-associated protein-2 (MAP2, anti-MAP2, 1:500, Chemicon); perivascular macrophages, ED2 (anti-ED2, 1:800, Serotec) were used to identify the type of P2X₄R-positive cells. Following incubation, tissue sections were washed and incubated for 3 hr at room temperature in the secondary antibody solution (anti-rabbit IgG conjugated Alexa Fluor™ 488 or anti-mouse IgG conjugated Alexa Fluor™ 546, 1:1000, Molecular Probes). The spinal cord sections were analysed using a MicroRadiance Confocal Imaging System (Bio-Rad) and an Olympus IX70 microscope equipped for epifluorescence. To assess immunofluorescence staining of cells quantitatively, we randomly selected dorsal horn fields displayed at high magnification. Microglia, as identified by OX42 immunofluorescence were outlined, and the immunofluorescence intensity (using a 9-bit scale) of the P2X₄R or OX42 was determined as the average pixel intensity within each cell. Background fluorescence intensity was determined and was subtracted from the value obtained for microglia.

Western Blotting

Western blot analysis of P2X₄R expression in the membrane fraction from L4-L6 spinal cord was performed using P2X₄R polyclonal antibody (Oncogene) as follows.

The membrane fraction from spinal cord segments L4-L6 ipsilateral or contralateral to the nerve injury was used. Twenty μg aliquots were subjected to 12.5% SDS-PAGE, and proteins were transferred electrophoretically to nitrocellulose membranes. After blocking, the membranes were incubated with anti-rat P2X₄R polyclonal antibody (1:200; Oncogene) and then were incubated with HRP-conjugated secondary antibody. The blots were detected using a chemiluminescence method (ECL system; Amersham) and exposed to autoradiography films (Hyperfilm-ECL; Amersham).

Microglial Culture

Rat primary cultured microglia were prepared according to the method described previously (Nakajima, K. et al. Identification of elastase as a secretory protease from cultured rat microglia. J Neurochem 58, 1401-1408. (1992)). In brief, mixed glial culture was prepared from neonatal Wistar rats and maintained for 10-16 days in DMEM with 10% fetal bovine serum. Immediately prior to experiments microglia were collected by a gentle shake as the floating cells over the mixed glial culture. The microglia were transferred to cover slips or to Eppendorf tubes for subsequent intrathecal administration.

Statistics

Statistical analyses of the results were evaluated using the Student's t test, the Student's paired t test or the Mann-Whitney U test.

Results

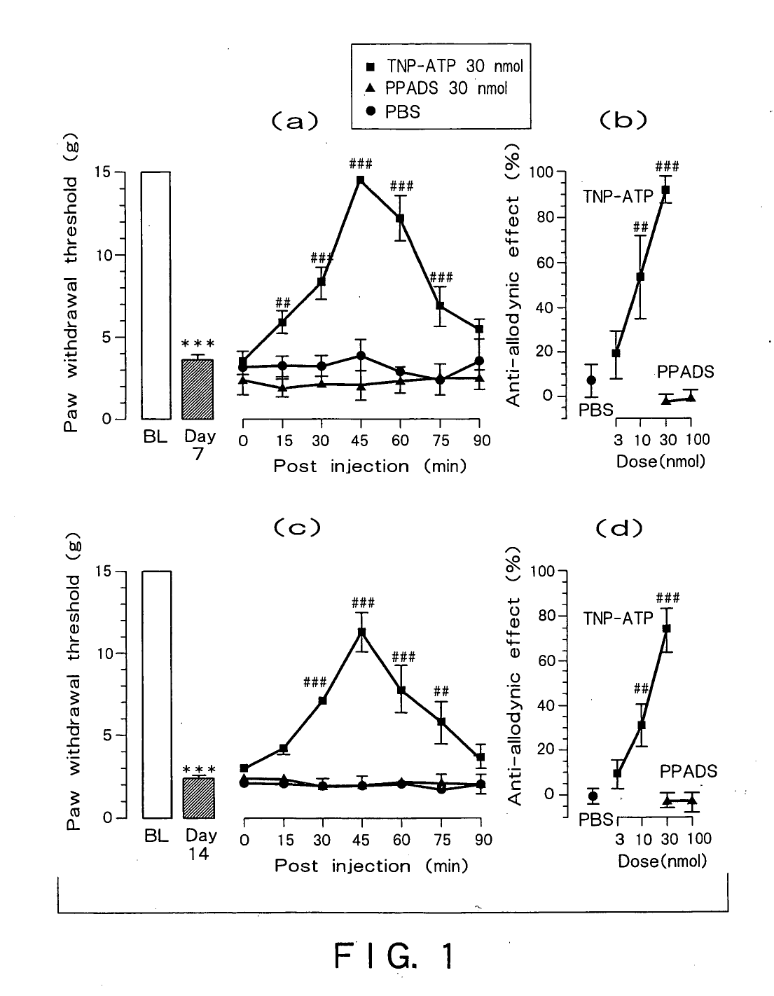

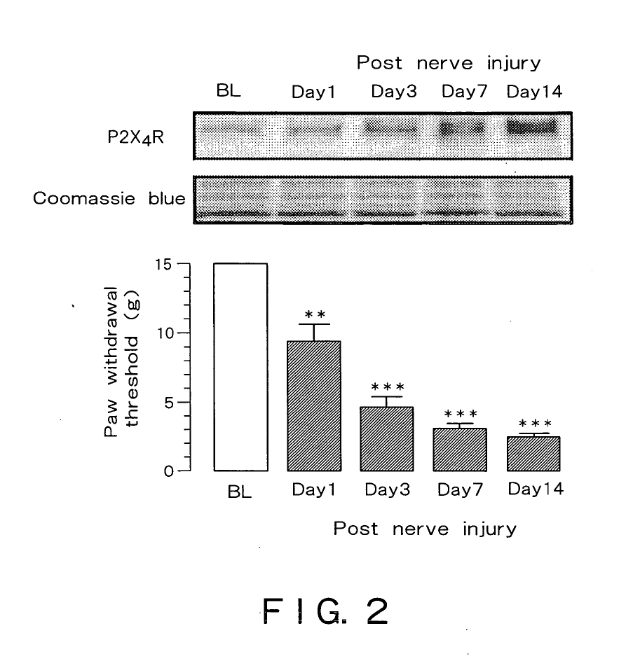

We investigated the mechanisms underlying tactile allodynia using a 5th lumbar (L5) spinal nerve injury model (Kim, S. H. & Chung, J. M. An experimental model for peripheral neuropathy produced by segmental spinal nerve ligation in the rat. Pain 50, 355-363. (1992)). Animals with L5 nerve injury but not sham controls (not illustrated) displayed marked tactile allodynia: paw withdrawal threshold decreased from 15.1±0.1 g (n=15 animals) before the injury to 3.1±0.3 g (n=15 animals) at day 7 (P<0.001, FIG. 1a) and 2.4±0.2 g (n=15 animals) at day 14 (P<0.001, FIG. 1c). We tested for involvement of P2X receptors (P2XRs) in the tactile allodynia by using TNP-ATP (Khakh, B. S. et al. International union of pharmacology. XXIV. Current status of the nomenclature and properties of P2X receptors and their subunits. Pharmacol Rev 53, 107-118. (2001); Virginio, C., Robertson, G., Surprenant, A. & North, R. A. Trinitrophenyl-substituted nucleotides are potent antagonists selective for P2X₁, P2X₃, and heteromeric P2X_{2/3} receptors. Mol Pharmacol 53, 969-973. (1998)), an antagonist of P2XR subtypes P2X₁₋₄R. We found that following intrathecal injection of TNP-ATP (30 nmol) paw withdrawal threshold increased gradually, peaked about 45 min after the injection and then returned to the pre-injection level over the subsequent 45 min. When TNP-ATP (30 nmol) was administered on day 7, the paw withdrawal threshold 45 min after injection was 14.6±0.5 g (n=6 animals; P<0.001). Paw withdrawal threshold at the peak of the effect of TNP-ATP was not different from that prior to nerve injury, and thus, tactile allodynia was reversed by TNP-ATP (30 nmol) on day 7. On day 14, paw withdrawal threshold was 11.6±1.3 g (n=6 animals) 45 min after injecting TNP-ATP (P<0.001), which represented 73% recovery of paw withdrawal threshold. Intrathecal administration of the vehicle (phosphate buffered saline —PBS) had no effect on either testing day (FIG. 1a-d). The increase in paw withdrawal threshold by TNP-ATP was dose-dependent with that producing half-maximum effect calculated as 8.1 nmol on day 7 and 15.5 nmol on day 14 (FIG. 1b,d). We observed no alteration in motor behaviour following TNP-ATP administration (data not illustrated). Nor did TNP-ATP affect paw withdrawal threshold on the side contralateral to the nerve injury: 45 min after PBS the threshold was 15.1±0.1 g compared with 14.3±0.8 g 45 min after TNP-ATP 30 nmol at 45 min (P>0.1). These results together indicate that TNP-ATP caused a dose-dependent, reversible recovery of paw withdrawal threshold on the nerve-injured side without a non-specific effect on motor or sensory functioning. Thus, tactile allodynia produced by nerve injury appears to be mediated at the spinal level by a P2XR sensitive to TNP-ATP.

We next tested PPADS, an antagonist of P2XR subtypes P2X_{1,2,3,5,7}R but not of P2X₄R, that has antinociceptive effects in models of acute and inflammatory pain (Tsuda, M., Ueno, S. & Inoue, K. Evidence for the involvement of spinal endogenous ATP and P2X receptors in nociceptive responses caused by formalin and capsaicin in mice. Br J Pharmacol 128, 1497-1504. (1999); Zheng, J. H. & Chen, J. Modulatory roles of the adenosine triphosphate P2X-purinoceptor in generation of the persistent nociception induced by subcutaneous bee venom injection in the conscious rat. Neurosci Lett 278, 41-44. (2000)). We found that intrathecal administration of PPADS (30 or 100 nmol) had no effect on paw withdrawal threshold on either day 7 (FIG. 1a, b) or day 14 (FIG. 1c, d). At these intrathecal doses, PPADS is known to suppress nociceptive behaviours caused by intrathecal injection of the P2X_{1,3}R agonist α,β-methylene ATP (Tsuda, M., Ueno, S. & Inoue, K. In vivo pathway of thermal hyperalgesia by intrathecal administration of a,b-methylene ATP in mouse spinal cord: involvement of the glutamate-NMDA receptor system. Br J Pharmacol 127, 449-456. (1999)), and those during the second phase of the formalin test (Tsuda, M., Ueno, S. & Inoue, K. Evidence for the involvement of spinal endogenous ATP and P2X receptors in nociceptive responses caused by formalin and capsaicin in mice. Br J Pharmacol 128, 1497-1504. (1999)) or evoked by injection of bee venom (Zheng, J. H. & Chen, J. Modulatory roles of the adenosine triphosphate P2X-purinoceptor in generation of the persistent nociception induced by subcutaneous bee venom injection in the conscious rat. Neurosci Lett 278, 41-44. (2000)), both models of inflammatory pain. Thus, these are doses of PPADS that should have been sufficient to increase paw withdrawal threshold in the present study if the P2XRs involved in tactile allodynia were PPADS-sensitive. The lack of effect of PPADS on paw withdrawal threshold together with the increase by TNP-ATP indicates that tactile allodynia caused by L5 nerve injury depends upon spinal P2XRs that are sensitive to TNP-ATP and insensitive to PPADS. The pharmacological profile of these P2XRs is consistent with that of the P2X₄R subtype (Bo, X., Zhang, Y, Nassar, M., Burnstock, G. & Schoepfer, R. A P2X purinoceptor cDNA conferring a novel pharmacological profile. FEBS Lett 375, 129-133. (1995); Buell, G., Lewis, C., Collo, G., North, R. A. & Surprenant, A. An antagonist-insensitive P2X receptor expressed in epithelia and brain. EMBO J. 15, 55-62. (1996); Seguela, P., Haghighi, A., Soghomonian, J. J. & Cooper, E. A novel neuronal P2X ATP receptor ion channel with widespread distribution in the brain. J Neurosci 16, 448-455. (1996); Soto, F. et al. P2×4: an ATP-activated ionotropic receptor cloned from rat brain. Proc Natl Acad Sci USA 93, 3684-3688. (1996); Wang, C. Z., Namba, N., Gonoi, T, Inagaki, N. & Seino, S. Cloning and pharmacological characterization of a fourth P2X receptor subtype widely expressed in brain and peripheral tissues including various endocrine tissues. Biochem Biophys Res Commun 220, 196-202. (1996); Khakh, B. S. et al. International union of pharmacology. XXIV. Current status of the nomenclature and properties of P2X receptors and their subunits. Pharmacol Rev 53, 107-118. (2001)) and therefore, we further explored the role of P2X₄Rs in tactile allodynia following nerve injury.

We examined the level of P2X₄R protein in homogenates from spinal cord of naive and nerve-injured rats and found that P2X₄R protein in the ipsilateral spinal cord increased dramatically after L5 nerve injury (FIG. 2): the increase in P2X₄R was detected as early as day 1 and the highest level was observed on day 14. In contrast, the level of P2X₄R protein in the contralateral spinal cord was not different on either day 7 or day 14 as compared with naive rats (not illustrated). The time-course of the change in P2X₄R level in the spinal cord and the bilateral difference in P2X₄R levels match the emergence of the tactile allodynia (FIG. 2, lower). In order to examine the distribution of P2X₄R, we did immunofluorescence on sections of the L5 spinal dorsal horn. In the spinal cord ipsilateral to the nerve injury, we observed strong, punctate P2X₄R immunofluorescence dotted throughout the dorsal horn; the punctate labelling observed at low magnification was due to immunofluorescence of individual cells (see below). In contrast, P2X₄R immunofluorescence was weaker and much less extensive in the dorsal horn contralateral to the nerve injury or of sham-operated rats. The immunofluorescence was abolished by preabsorbing the antibody with the immunogen peptide for P2X₄R antibody, indicating that the observed staining was not a non-specific signal. Thus, the level of P2X₄Rs increases dramatically in the dorsal horn ipsilateral to the nerve injury with a time course matching that of the development of tactile allodynia. In contrast to this increase in P2X₄R level, we found that there was no change in P2X₄R immunofluorescence in the dorsal horn 7 days after intraplantar injection of complete Freund's adjuvant (CFA). CFA produces sustained inflammation of the paw and prolonged pain hypersensitivity. Thus, nerve injury but not persistent peripheral inflammation caused an increase in P2X₄R level in the dorsal horn.

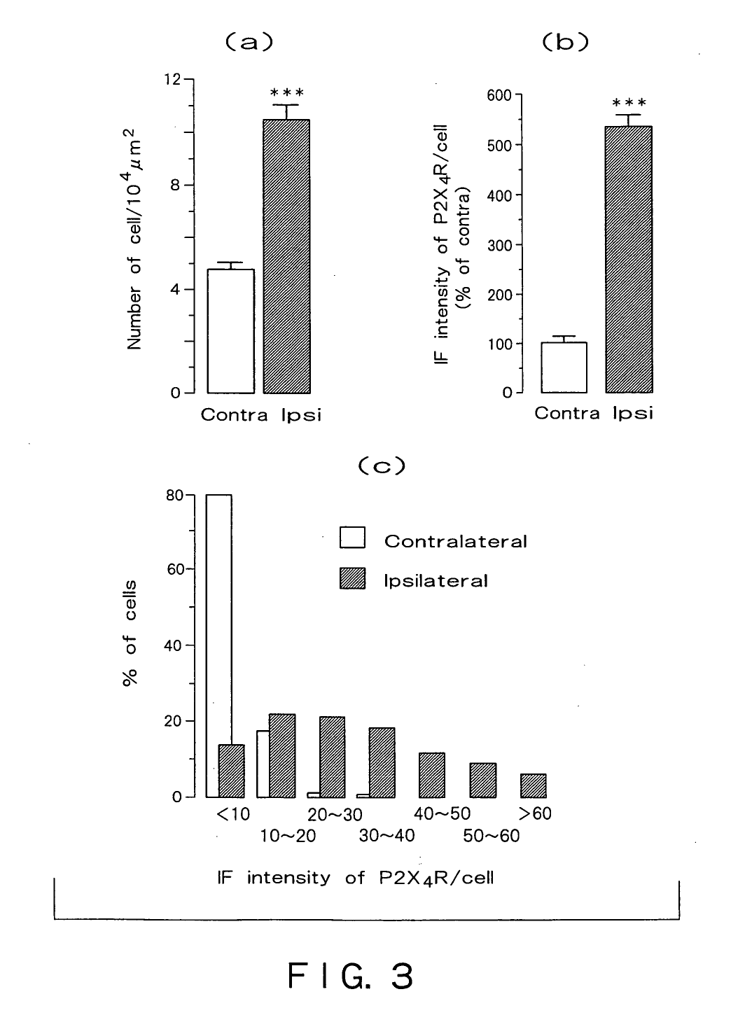

To identify the type of cell expressing P2X₄Rs after nerve injury, on day 14 we carried out double immunofluorescence labelling for P2X₄R and for cell type-specific markers: for neurons, microtubule-associated protein 2 (MAP2) and neuronal nuclei (NeuN); for astrocytes, glial fibrillary acidic protein (GFAP); or for microglia, OX42 (Honore, P. et al. Murine models of inflammatory, neuropathic and cancer pain each generates a unique set of neurochemical changes in the spinal cord and sensory neurons. Neuroscience 98, 585-598 (2000)). We found that cells showing P2X₄R immunofluorescence were not double-labelled for MAP2, NeuN or GFAP. Rather, almost all of P2X₄R-positive cells were double-labelled with OX42 (385 of. 395 P2X₄R-positive cells examined), indicating that P2X₄Rs were expressed in microglia, but not in neurons or astrocytes. OX42 recognizes the complement receptor type 3 (CR3), expression of which is greatly increased in hyperactive versus resting microglia (Aldskogius, H. & Kozlova, E. N. Central neuron-glial and glial-glial interactions following axon injury. Prog Neurobiol 55, 1-26. (1998)). We found that OX42 labelling was greater in the dorsal horn ipsilateral to the nerve injury whereas OX42 labelling in the dorsal horn was low bilaterally in sham-operated animals. OX42-positive cells were more numerous (FIG. 3a) and displayed hypertrophic morphology in the dorsal horn on the side of the nerve injury as compared with the contralateral side. These results indicate that nerve injury induced a switch from the resting to the hyperactive phenotype in the population of microglia in the dorsal horn. We found that cells expressing P2X₄R immunofluorescence in the ipsilateral dorsal horn showed high levels of OX42 labelling. The mean level of intensity of P2X₄R immunofluorescence per OX42-positive cell was on average 5.4-fold higher in the ipsilateral as compared with the contralateral dorsal horn (P<0.001, FIG. 3b) and the distribution of P2X₄R immunofluorescence intensities per OX42-positive cell was skewed to the right (FIG. 3c). Therefore, we concluded that in the dorsal horn following nerve injury hyperactive microglia are the cell type which express P2X₄R and that the level of P2X₄R expression is dramatically increased in individual hyperactive microglia. Moreover, we observed that microglia in primary culture, cells which show the hyperactive phenotype, express functional P2X₄Rs that are activated by ATP (50 μM) and inhibited by TNP-ATP (10 μM) but not by PPADS (10 μM; see Supplementary Information 1).

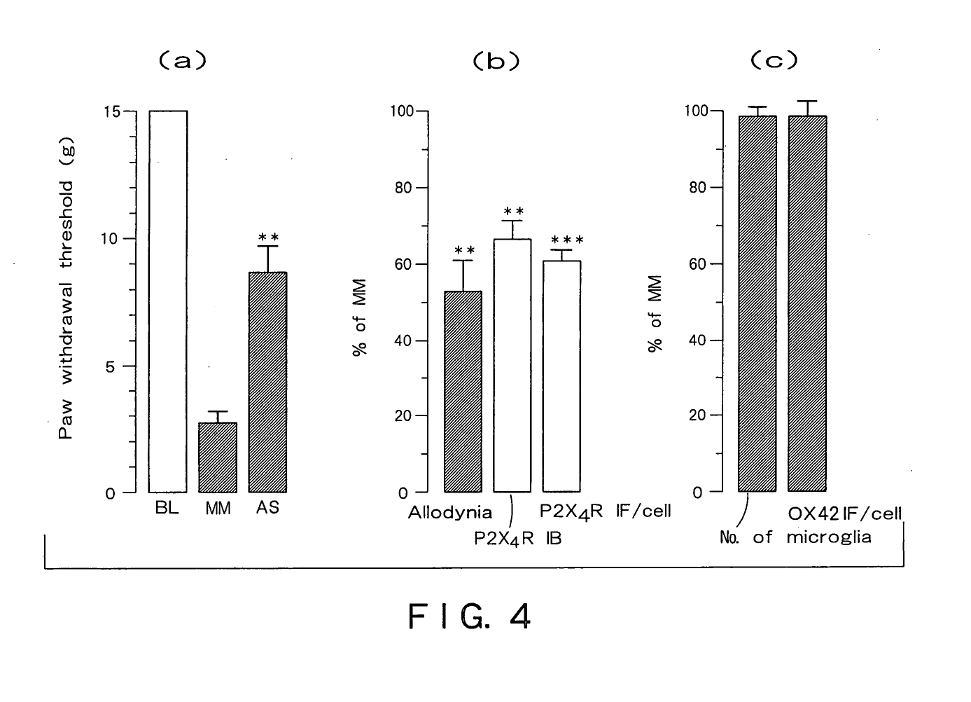

Because we find that the expression of P2X₄Rs is markedly upregulated in individual microglia following nerve injury, the results above imply that tactile allodynia following nerve injury is critically dependent upon functional P2X₄Rs in hyperactive microglia in the dorsal horn. We predicted therefore that suppressing expression of P2X₄Rs should prevent tactile allodynia following nerve injury. We tested this by means of intrathecal treatment with an antisense oligodeoxynucleotide (ODN) targeting P2X₄R or with a mismatch ODN as a control. The animals were treated for 7 days beginning on the day of the nerve lesion. We found that the nerve injury-induced decrease in paw withdrawal threshold was significantly less in animals treated with P2X₄R antisense ODN (5 nmol, n=11 animals) as compared with that in animals treated with mismatch ODN (5 nmol, n=10 animals) (P<0.01, FIG. 4a); paw withdrawal threshold in animals treated with mismatch ODN was not different from that of untreated controls (FIG. 1a, P>0.48). Paw withdrawal threshold in animals treated with P2X₄R antisense ODN recovered by 47.4±8.3% towards the baseline level prior to nerve injury (FIG. 4b). Furthermore, we found that the level of P2X₄R protein in homogenates from the spinal cord of antisense ODN-treated rats (n=5 animals) was 32.0±4.8% less than that of mismatch ODN-treated rats (n=4 animals; P<0.01, FIG. 4b). Also, the immunofluorescence intensity of P2X₄R protein in individual microglia in the dorsal horn of antisense ODN-treated rats (n=116 microglial cells) was significantly lower than that of mismatch ODN-treated rats (n=115 microglial cells; 37.1±0.2% decrease, P<0.001; FIG. 4b). However, we found that there was no difference in the number of microglia in the dorsal horn between antisense as compared with mismatch ODN-treated rats (FIG. 4c) nor was there a difference in the level of OX42 immunofluorescence per individual microglia. In addition, microglia maintained a hypertrophic morphology in animals treated with P2X₄R antisense. These results indicate that intrathecal treatment with P2X₄R antisense ODN suppressed both the tactile allodynia and the increase in P2X₄R expression following nerve injury but P2X₄R antisense ODN treatment did not suppress OX42 labelling nor prevent the switch to the hyperactive phenotype of the microglia in the dorsal horn.

We next investigated whether activation of P2X₄Rs in hyperactive microglia is sufficient to produce tactile allodynia. We tested paw withdrawal threshold in normal rats after intrathecal injection of microglia which had been grown in primary culture (FIG. 5a), and which therefore expressed P2X₄Rs and high levels of OX42. The microglia were pre-incubated for 1 hr with PBS without or with ATP (50 μM) to preferentially activate P2X₄Rs but not P2X₇Rs (see Supplementary Information 1). We found that paw withdrawal threshold dramatically decreased after intrathecal administration of ATP-stimulated microglia (n=7 animals; FIG. 5b). In contrast, paw withdrawal threshold was unaffected by intrathecal administration of PBS-treated microglia (n=5 animals), PBS alone (n=4 animals) or ATP (50 μM) alone (n=4 animals; FIG. 5b). The decrease in paw withdrawal threshold was prevented by including TNP-ATP (10 μM) together with ATP in the pre-incubation (n=5 animals; FIG. 5b). Thus, we concluded that P2X₄R-stimulated microglia were sufficient to produce tactile allodynia in otherwise naive rats.

The decrease in paw withdrawal threshold developed progressively over a 5 hr period after administering the ATP-stimulated microglia (FIG. 5b) and therefore, we wondered whether stimulation of P2X₄Rs was required at the time when the allodynia was observed. We investigated this by intrathecal injection of TNP-ATP (30 nmol) 5 hr after the ATP-stimulated microglia had been administered (n=5 animals; FIG. 5c). We found that paw withdrawal threshold increased gradually after the injection of TNP-ATP, peaking 45-75 min after the injection (45 and 60 min: P<0.05, 75 min: P<0.01 vs. prior to TNP-ATP). Paw withdrawal threshold at the peak of the effect of TNP-ATP was not different from the baseline level prior to intrathecal injection of the ATP-stimulated microglia indicating that TNP-ATP reversed the allodynia caused by the ATP-stimulated microglia. Importantly, by 90 min after injecting TNP-ATP paw withdrawal threshold had significantly decreased, implying reversal of the effect of TNP-ATP and recovery of the tactile allodynia induced by administering the ATP-stimulated microglia. Taking these results together we conclude that stimulation of P2X₄Rs is required when tactile allodynia caused by ATP-stimulated microglia is observed and this tactile allodynia is therefore like that caused by peripheral nerve injury. Furthermore, these results suggest that P2X₄Rs stimulation of microglia is not only sufficient to induce tactile allodynia but also is necessary for maintaining the allodynia.

Supplementary Information 1

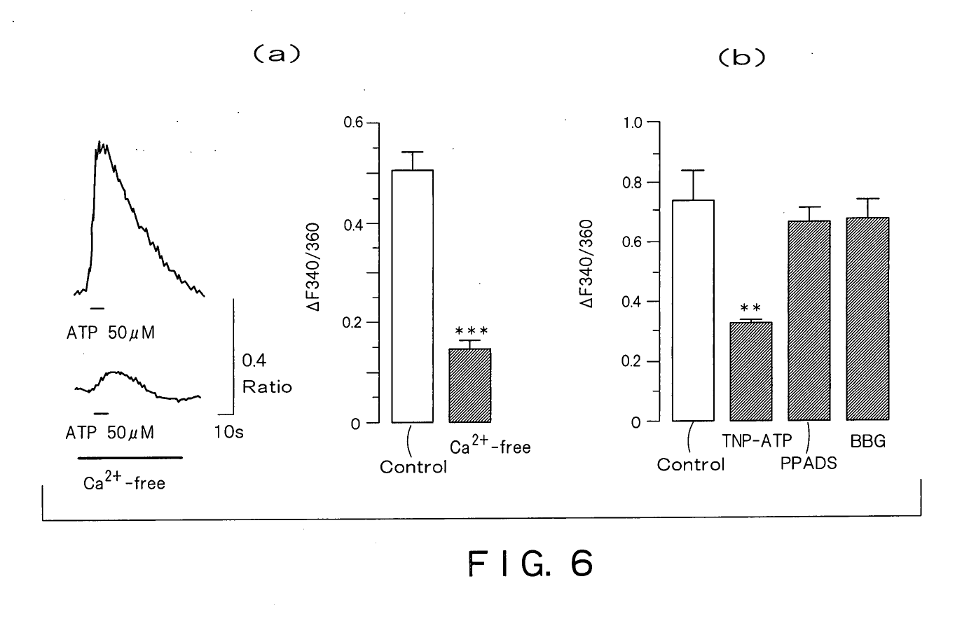

Functional P2X₄R is expressed in hyperactive microglia in primary culture. In order to determine whether P2X₄Rs are functional in microglia with the hyperactive phenotype, we studied microglia in primary culture (Nakajima, K. et al. Identification of elastase as a secretory protease from cultured rat microglia. J Neurochem 58, 1401-1408, 1992) where all of the cells express high levels of OX42. We tested for expression of mRNA encoding P2XRs by using reverse transcriptase-polymerase chain reaction (RT-PCR) with primer pairs specific for each of the 7 subtypes of P2XR (P2X₁R-P2X₇R). RT-PCR reaction product was detected for P2X₄R as well as for P2X₇R, a P2XR subtype previously reported to be expressed in microglia (Ferrari, D. et al. Mouse microglial cells express a plasma membrane pore gated by extracellular ATP. J Immunol 156, 1531-1539, 1996), whereas no RT-PCR product was detected for the other P2XR subtypes. To determine whether P2X₄R protein was expressed, we used immunocytochemistry and observed intense immunofluorescence for P2X₄R. Thus, microglia in culture express both mRNA and protein for P2X₄R. As P2X₄Rs are reported to be highly permeable to Ca²⁺ (Khakh, B. S. et al. International union of pharmacology. XXIV. Current status of the nomenclature and properties of P2X receptors and their subunits. Pharmacol Rev 53, 107-118, 2001), we investigated whether the P2X₄Rs on these microglia were functional by applying the agonist ATP and monitoring the level of intracellular Ca²⁺ ([Ca²⁺]i) in individual cells using the Ca²⁺-sensitive fluorescent dye fura-2. We found that ATP (50 μM, 10 s) produced a transient increase in the 340/360 emission ratio for fura-2 (n=28 cells), indicating that ATP caused an increase [Ca²⁺]i in the microglia (FIG. 6a). When the extracellular solution had no added Ca²⁺ the increase in 340/360 emission ratio evoked by ATP was greatly blunted (n=28 cells, P<0.001). The ATP-evoked increase in 340/360 ratio was suppressed by TNP-ATP (n=14 cells, P<0.01) but was unaffected by PPADS (n=26 cells, FIG. 6d). In addition, the effect of ATP (50 μM) was not altered by brilliant blue G (BBG, 100 nM, n=21 cells), which is known to differentially block P2X₇R but not other subtypes of P2XR (Jiang, L. H., Mackenzie, A. B., North, R. A. & Surprenant, A. Brilliant blue G selectively blocks ATP-gated rat P2X₇ receptors. Mol Pharmacol 58, 82-88, 2000). Together, these results indicate that microglia in primary culture, which like hyperactive microglia in situ show high levels of OX42, express functional P2X₄Rs.

The detailed procedures are as follows. Rat primary cultured microglia were prepared according to the method described previously (Nakajima, K. et al. Identification of elastase as a secretory protease from cultured rat microglia. J Neurochem 58, 1401-1408, 1992). In brief, mixed glial culture was prepared from neonatal Wistar rats and maintained for 10-16 days in DMEM with 10% fetal bovine serum. Microglia were obtained as floating cells over the mixed glial culture. The floating cells were collected by a gentle shake and transferred to appropriate dishes or glasses, and then the microglia attached to them were used for RT-PCR, intracellular Ca²⁺ imaging, immunocytochemistry. RT-PCR was carried out as described previously (Shigemoto-Mogami, Y et al. Mechanisms underlying extracellular ATP-evoked interleukin-6 release in mouse microglial cell line, MG-5. J Neurochem 78, 1339-1349, 2001). Microglial cells (primary culture) were directly lysed with 0.5 ml of RNA STAT-60 (Tel-Test “B” Inc.) and total RNA was isolated. Reverse transcription was performed with 1 μg of total RNA using M-MLV reverse transcriptase. One μl of the RT product was added to the reaction mixture containing 1×PCR buffer (10 mM Tris-HCl, 50 mM KCl, pH 8.3), 1.5 mM MgCl₂, 0.2 mM dNTPs, 2.5 units of Taq polymerase, and P2X₁-P2X₇ receptors specific primers;
P2X₁R (296 bp): 5′-TCTTCTTCGTGAGGCTGAGA-3′ (SEQ ID NO: 5) and 5′-ACTGGTAGATGGGTTTGCAG-3′ (SEQ ID NO: 6),
P2X₂R (358 bp): 5′-GAATCAGAGTGCAACCCCAA-3′ (SEQ ID NO: 7) and 5′-TCACAGGCCATCTACTTGAG-3′ (SEQ ID NO: 8),
P2X₃R (462 bp): 5′-AAGTACCGCTGTGTGTCTGA-3′ (SEQ ID NO: 9) and 5′-ATCCAGCCGAGTGAAGGAAT-3′ (SEQ ID NO: 10),
P2X₄R (515 bp): 5′-TCACCACGTCCTACCTCAAA-3′ (SEQ ID NO: 11) and 5′-CTGCTCGTAGTCTTCCACAT-3′ (SEQ ID NO: 12),
P2X₅R (485 bp): 5′-ACACACACACTCCATCTCCT-3′ (SEQ ID NO: 13) and 5′-CTGCTTCACGTTCACAATGG-3′ (SEQ ID NO: 14),
P2X₆R (411 bp): 5′-TMGGAACTGGAGAAACGGC-3′ (SEQ ID NO: 15) and 5′-TAGGTGTTGTCCCAGGTATC-3′ (SEQ ID NO: 16),
P2X₇R (497 bp): 5′-TAGTACACGGCATCTTCGAC-3′ (SEQ ID NO: 17) and 5′-CTGAACTGCCACCTCTGTAA-3′ (SEQ ID NO: 18).
   After PCR amplification, the products were analysed by electrophoresis on agarose gel with ethidium bromide. Single-cell fluorescence monitoring of intracellular Ca²⁺. [Ca²⁺]i in single microglial cells was monitored by using the Ca²⁺ sensitive fluorescent dye fura-2. The microglia were incubated with 10 μM fura-2 acetoxymethylester for 45 min in DMEM. Then, the microglia were washed with balanced salt solution (BSS; composition in mM: NaCl 150, KCl 5, CaCl₂ 1.8, MgCl₂ 1.2, D-glucose 10 and HEPES 25; pH 7.4) and mounted on an inverted fluorescence microscope equipped with a Xenon-lamp and band-pass filters of 340 nm and 360 nm wavelength. The emission fluorescence was measured at 510 nm. Image data, recorded by a high-sensitivity silicon intensifier target camera, were processed by a Ca²⁺-analyzing system (Furusawa Lab. Appliance. Co.). ATP (50 μM) was applied for 10 s. TNP-ATP (10 μM), PPADS (10 μM) and BBG (100 nM) were applied for 10 min before and during ATP application.
   Supplementary Information 2

ATP levels in the cerebrospinal fluid (CSF) from the lumbar spinal cord is not changed following nerve injury. The ATP levels in the CSF collected from the lumbar spinal cord were measured by a luciferin-luciferase bioluminescence assay. We found that the level of ATP in CSF was not different in nerve-injured rats (54.3±14.8 nM, n=4 animals) as compared with naive controls (58.1±12.4 nM, n=4 animals). Inasmuch as the lack of change in the CSF ATP level indicates that extracellular ATP levels at sites of action at P2X₄Rs on microglia within the dorsal horn are unchanged after nerve injury, we may infer that tactile allodynia following nerve injury depends upon the enhanced expression of P2X₄Rs which are then activated by the constitutive level of the endogenous ligand ATP.

1. A method of identifying a compound useful for the treatment or prevention of tactile allodynia induced after nerve injury, comprising:
(a) contacting a cell expressing P2X₄ receptor on the surface thereof, with a test compound, in the presence of P2X₄ receptor agonist,
(b) determining whether or not said test compound inhibits an interaction of said P2X₄ receptor agonist and P2X₄ receptor on the surface of the cell, and
(c) identifying the test compound which inhibits said interaction, as useful for the treatment or prevention of tactile allodynia induced after nerve injury.

2. (Cancel)

3. The method according to claim 1, wherein the cell is mammalian cell.

4. The method according to claim 1, wherein the cell does not express any P2X receptors other than P2X₄ receptor.

5. The method according to claim 1, wherein the P2X₄ receptor agonist is ATP or ADP.

6. The method according to claim 1, wherein the contacting step (a) comprises incubating the cell and the test compound in the absence of the P2X₄ receptor agonist, and then incubating them in the presence of the P2X₄ receptor agonist.

7. The method according to claim 1, wherein the determining step (b) comprises measuring P2X₄ receptor-mediated ion flux of at least one ion selected from the group consisting of Na⁺, K⁺, and Ca²⁺.

8. The method according to claim 7, wherein the contacting step (a) is carried out in the presence of the ion.

9. The method according to claim 1, wherein the determining step (b) comprises comparing intensity of the interaction with that of control sample obtained in the absence of any test compounds.

10. A method of identifying a compound useful for the treatment or prevention of neuropathic pain, comprising:
(a) contacting a microglia in inactive-form with a test compound, in the presence of microglia-activator,
(b) determining whether or not said test compound inhibits an activation of said microglia, and
(c) identifying the test compound which inhibits said activation, as useful for the treatment or prevention of neuropathic pain.

11. The method according to claim 10, wherein the neuropathic pain is tactile allodynia induced after nerve injury.

12. The method according to claim 10, wherein the microglia-activator is ATP or ADP.

13. The method according to claim 10, wherein the contacting step (a) comprises incubating the cell and the test compound in the absence of the microglia-activator, and then incubating them in the presence of the microglia-activator.

14. A pharmaceutical composition comprising a P2X₄ receptor inhibitor and a pharmaceutically acceptable carrier.

15. The pharmaceutical composition according to claim 14 for use in treatment or prevention of neuropathic pain.

16. The pharmaceutical composition according to claim 15, wherein the neuropathic pain is tactile allodynia induced after nerve injury.

17. The pharmaceutical composition according to claim 14, wherein the P2X₄ receptor inhibitor is a P2X₄ receptor antagonist.

18. The pharmaceutical composition according to claim 14, wherein the P2X₄ receptor inhibitor is an antibody or an antibody fragment which binds to P2X₄ receptor protein on the cell surface and prevents the interaction between the receptor and its agonist.

19. The pharmaceutical composition according to claim 14, wherein the P2X₄ receptor inhibitor is an antisense nucleic acid molecule that specifically suppresses expression of P2X₄ receptor gene.

20. The pharmaceutical composition according to claim 14, wherein the P2X₄ receptor inhibitor is an siRNA nucleic acid molecule that specifically suppresses expression of P2X₄ receptor gene.

21. The pharmaceutical composition according to claim 14, wherein the P2X₄ receptor inhibitor is a vector expressing an siRNA nucleic acid molecule that specifically suppresses expression of P2X₄ receptor gene.

22. A pharmaceutical composition comprising a microglial activation-inhibitor and a pharmaceutically acceptable carrier.

23. The pharmaceutical composition according to claim 22 for use in treatment or prevention of neuropathic pain.

24. The pharmaceutical composition according to claim 23, wherein the neuropathic pain is tactile allodynia induced after nerve injury.

25. The pharmaceutical composition according to claim 22, wherein the microglial activation-inhibitor is a P2Y₁₂ receptor inhibitor.

26. A method for treating or preventing neuropathic pain comprising administering to a subject a therapeutically effective amount of P2X₄ receptor inhibitor.

27. The method according to claim 26, wherein the neuropathic pain is tactile allodynia induced after nerve injury.

28. The method according to claim 26, wherein the P2X₄ receptor inhibitor is a P2X₄ receptor antagonist.

29. The method according to claim 26, wherein the P2X₄ receptor inhibitor is an antibody or an antibody fragment which binds to P2X₄ receptor protein on the cell surface and prevents the interaction between the receptor and its agonist.

30. The method according to claim 26, wherein the P2X₄ receptor inhibitor is an antisense nucleic acid molecule that specifically suppresses expression of P2X₄ receptor gene.

31. The method according to claim 26, wherein the P2X₄ receptor inhibitor is an siRNA nucleic acid molecule that specifically suppresses expression of P2X₄ receptor gene.

32. The method according to claim 26, wherein the P2X₄ receptor inhibitor is a vector expressing an siRNA nucleic acid molecule that specifically suppresses expression of P2X₄ receptor gene.

33. The method according to claim 26, wherein the P2X₄ receptor inhibitor is administered intraspinally.

34. The method according to claim 33, wherein the P2X₄ receptor inhibitor is administered by intrathecal injection.

35. The method according to claim 26, wherein the P2X₄ receptor inhibitor is administered in admixture with a pharmaceutically acceptable carrier.

36. A method for treating or preventing neuropathic pain comprising administering to a subject a therapeutically effective amount of microglial activation-inhibitor.

37. The method according to claim 36, wherein the neuropathic pain is tactile allodynia induced after nerve injury. 5.

38. The method according to claim 36, wherein the microglial activation-inhibitor is a P2Y₁₂ receptor inhibitor.

39. The method according to claim 36, wherein the microglial activation-inhibitor is administered intraspinally.

40. The method according to claim 39, wherein the microglial activation-inhibitor is administered by intrathecal injection.

41. The method according to claim 36, wherein the microglial activation-inhibitor is administered in admixture with a pharmaceutically acceptable carrier.


SEQUENCE LISTING (20 sequences)

SEQ ID NO: 1 - DNA, 1167 bp, HOMO SAPIENS
  CDS (1)..(1164)
atggcgggctgctgctccgcgctggcggccttcctgttcgagtacgacacgccgcgcatcgtgctcatccgcagccgcaaagtggggctcatgaaccgcgccgtgcaactgctcatcctggcctacgtcatcgggtgggtgtttgtgtgggaaaagggctaccaggaaactgactccgtggtcagctccgttacgaccaaggtcaagggcgtggctgtgaccaacacttctaaacttggattccggatctgggatgtggcggattatgtgataccagctcaggaggaaaactccctcttcgtcatgaccaacgtgatcctcaccatgaaccagacacagggcctgtgccccgagattccagatgcgaccactgtgtgtaaatcagatgccagctgtactgccggctctgccggcacccacagcaacggagtctcaacaggcaggtgcgtagctttcaacgggtccgtcaagacgtgtgaggtggcggcctggtgcccggtggaggatgacacacacgtgccacaacctgcttttttaaaggctgcagaaaacttcactcttttggttaagaacaacatctggtatcccaaatttaatttcagcaagaggaatatccttcccaacatcaccactacttacctcaagtcgtgcatttatgatgctaaaacagatcccttctgccccatattccgtcttggcaaaatagtggagaacgcaggacacagtttccaggacatggccgtggagggaggcatcatgggcatccaggtcaactgggactgcaacctggacagagccgcctccctctgcttgcccaggtactccttccgccgcctcgatacacgggacgttgagcacaacgtatctcctggctacaatttcaggtttgccaagtactacagagacctggctggcaacgagcagcgcacgctcatcaaggcctatggcatccgcttcgacatcattgtgtttgggaaggcagggaaatttgacatcatccccactatgatcaacatcggctctggcctggcactgctaggcatggcgaccgtgctgtgtgacatcatagtcctctactgcatgaagaaaagactctactatcgggagaagaaatataaatatgtggaagattacgagcagggtcttgctagtgagctggaccagtga

SEQ ID NO: 2 - protein, 388 aa, HOMO SAPIENS
MAGCCSALAAFLFEYDTPRIVLIRSRKVGLMNRAVQLLILAYVIGWVFVWEKGYQETDSVVSSVTTKVKGVAVTNTSKLGFRIWDVADYVIPAQEENSLFVMTNVILTMNQTQGLCPEIPDATTVCKSDASCTAGSAGTHSNGVSTGRCVAFNGSVKTCEVAAWCPVEDDTHVPQPAFLKAAENFTLLVKNNIWYPKFNFSKRNILPNITTTYLKSCIYDAKTDPFCPIFRLGKIVENAGHSFQDMAVEGGIMGIQVNWDCNLDRAASLCLPRYSFRRLDTRDVEHNVSPGYNFRFAKYYRDLAGNEQRTLIKAYGIRFDIIVFGKAGKFDIIPTMINIGSGLALLGMATVLCDIIVLYCMKKRLYYREKKYKYVEDYEQGLASELDQ

SEQ ID NO: 3 - DNA, 16 bp, ARTIFICIAL
  Oligonucleotide
cagcccgccatggctc

SEQ ID NO: 4 - DNA, 16 bp, ARTIFICIAL
  Oligonucleotide
accgccgccagtgcct

SEQ ID NO: 5 - DNA, 20 bp, ARTIFICIAL
  Primer
tcttcttcgtgaggctgaga

SEQ ID NO: 6 - DNA, 20 bp, ARTIFICIAL
  Primer
actggtagatgggtttgcag

SEQ ID NO: 7 - DNA, 20 bp, ARTIFICIAL
  Primer
gaatcagagtgcaaccccaa

SEQ ID NO: 8 - DNA, 20 bp, ARTIFICIAL
  Primer
tcacaggccatctacttgag

SEQ ID NO: 9 - DNA, 20 bp, ARTIFICIAL
  Primer
aagtaccgctgtgtgtctga

SEQ ID NO: 10 - DNA, 20 bp, ARTIFICIAL
  Primer
atccagccgagtgaaggaat

SEQ ID NO: 11 - DNA, 20 bp, ARTIFICIAL
  Primer
tcaccacgtcctacctcaaa

SEQ ID NO: 12 - DNA, 20 bp, ARTIFICIAL
  Primer
ctgctcgtagtcttccacat

SEQ ID NO: 13 - DNA, 20 bp, ARTIFICIAL
  Primer
acacacacactccatctcct

SEQ ID NO: 14 - DNA, 20 bp, ARTIFICIAL
  Primer
ctgcttcacgttcacaatgg

SEQ ID NO: 15 - DNA, 20 bp, ARTIFICIAL
  Primer
taaggaactggagaaacggc

SEQ ID NO: 16 - DNA, 20 bp, ARTIFICIAL
  Primer
taggtgttgtcccaggtatc

SEQ ID NO: 17 - DNA, 20 bp, ARTIFICIAL
  Primer
tagtacacggcatcttcgac

SEQ ID NO: 18 - DNA, 20 bp, ARTIFICIAL
  Primer
ctgaactgccacctctgtaa

SEQ ID NO: 19 - DNA, 1029 bp, HOMO SAPIENS
  CDS (1)..(1026)
atgcaagccgtcgacaacctcacctctgcgcctggtaacaccagtctgtgcaccagagactacaaaatcacccaggtcctcttcccactgctctacactgtcctgttttttgttggacttatcacaaatggcctggcgatgaggattttctttcaaatccggagtaaatcaaactttattatttttcttaagaacacagtcatttctgatcttctcatgattctgacttttccattcaaaattcttagtgatgccaaactgggaacaggaccactgagaacttttgtgtgtcaagttacctccgtcatattttatttcacaatgtatatcagtatttcattcctgggactgataactatcgatcgctaccagaagaccaccaggccatttaaaacatccaaccccaaaaatctcttgggggctaagattctctctgttgtcatctgggcattcatgttcttactctctttgcctaacatgattctgaccaacaggcagccgagagacaagaatgtgaagaaatgctctttccttaaatcagagttcggtctagtctggcatgaaatagtaaattacatctgtcaagtcattttctggattaatttcttaattgttattgtatgttatacactcattacaaaagaactgtaccggtcatacgtaagaacgaggggtgtaggtaaagtccccaggaaaaaggtgaacgtcaaagttttcattatcattgctgtattctttatttgttttgttcctttccattttgcccgaattccttacaccctgagccaaacccgggatgtctttgactgcactgctgaaaatactctgttctatgtgaaagagagcactctgtggttaacttccttaaatgcatgcctggatccgttcatctattttttcctttgcaagtccttcagaaattccttgataagtatgctgaagtgccccaattctgcaacatctctgtcccaggacaataggaaaaaagaacaggatggtggtgacccaaatgaagagactccaatgtaa

SEQ ID NO: 20 - protein, 342 aa, HOMO SAPIENS
MQAVDNLTSAPGNTSLCTRDYKITQVLFPLLYTVLFFVGLITNGLAMRIFFQIRSKSNFIIFLKNTVISDLLMILTFPFKILSDAKLGTGPLRTFVCQVTSVIFYFTMYISISFLGLITIDRYQKTTRPFKTSNPKNLLGAKILSVVIWAFMFLLSLPNMILTNRQPRDKNVKKCSFLKSEFGLVWHEIVNYICQVIFWINFLIVIVCYTLITKELYRSYVRTRGVGKVPRKKVNVKVFIIIAVFFICFVPFHFARIPYTLSQTRDVFDCTAENTLFYVKESTLWLTSLNACLDPFIYFFLCKSFRNSLISMLKCPNSATSLSQDNRKKEQDGGDPNEETPM